(19) United States
(12) Patent Application Publication
(10) Pub. No.: US 2026/0091154 A1
(43) Pub. Date: Apr. 2, 2026

(54) Anti-Pathogenic Nanoparticle and Ion Generator for Airborne Pathogen Neutralization

(71) Applicants: Kui-Chiu Kwok, Eden Prairie, MN (US); Ali Reza Vatine, Eden Prairie, MN (US)

(72) Inventors: Kui-Chiu Kwok, Eden Prairie, MN (US); Ali Reza Vatine, Eden Prairie, MN (US)

(21) Appl. No.: 19/348,624

(22) Filed: Oct. 2, 2025

Related U.S. Application Data
(60) Provisional application No. 63/702,484, filed on Oct. 2, 2024.

(51) Int. Cl. A61L 9/22; A61L 9/14; A61L 101/30

(52) U.S. Cl. CPC A61L 9/22; A61L 9/14; A61L 2101/30; A61L 2103/75; A61L 2202/14; A61L 2202/15

(57) ABSTRACT

The present invention relates to a system and method for generating and dispersing a mixture of nanoparticles and ions for neutralization of airborne pathogens in enclosed environments. A nebulizer introduces a salt precursor into a flame ionization stage, which produces a mixture of ions and nanoparticles at concentrations of at least 1.0×10\hat{ }12 particles per cubic centimeter of air. The nanoparticles have an average size of less than 10 nanometers, and at approximately equal proportions with the ions. The systems and methods of the present invention reduce the pathogen viability by neutralization, inactivation, and agglomeration, and operate substantially free of ozone and, in alternate embodiments, include a two-duct alternating filtration arrangement for efficiency. Methods of testing include introducing pathogen surrogates into a chamber, generating the mixture of nanoparticles and ions by flame ionization, dispersing the mixture, and analyzing samples using aerosol and microbiological characterization techniques.

### FIELD OF THE INVENTION

The present invention generally relates to the generation of nanoparticles and ions for biological neutralization, pathogen inactivation, air disinfection, and nanoparticle engineering. More specifically, it relates to the generation and controlled dispersion of anti-pathogenic nanoparticles and ions for the rapid inactivation or neutralization of airborne microorganisms in enclosed environments.

### BACKGROUND OF THE INVENTION

Airborne transmission of pathogens remains a persistent global health challenge, resulting in deleterious effects on human health and economic development. Viruses such as influenza, SARS-CoV-2, respiratory syncytial virus (RSV), and bacterial pathogens such as Mycobacterium tuberculosis, Bacillus anthracis, Clostridium botulinum, Streptococcus pneumoniae, bacterial and fungal spores, etc., are readily spread through aerosolized particles in indoor spaces. They present a persistent health threat, where transmission in enclosed spaces remains a critical driver of outbreaks such as the recent COVID-19 pandemic. Transmission and the associated risk of infection increase in densely populated environments. Conventional air-cleaning technologies have been employed in attempts to mitigate this risk, with much effort to develop effective means for viral deactivation indoors, but each is hindered by specific limitations.

Mechanical filtration (e.g., HEPA filters): High-efficiency particulate air (HEPA) filters capture airborne particles by forcing air through dense meshes. While widely adopted, these filters do not inactivate pathogens, but merely trap them until replacement. Pathogens remain viable on filter media, creating a risk of secondary contamination. Furthermore, filtration is inherently delayed, since air must circulate through the filter, allowing infectious aerosols to persist and spread in the room before removal.

Ultraviolet (UV) irradiation: UV-C light has germicidal effects and is deployed in certain hospitals and HVAC systems. However, UV disinfection requires direct line-of-sight exposure and sufficient dwell time, which aerosols moving in air currents rarely receive. UV lamps are also energy-intensive, require maintenance, and may pose safety risks to occupants due to accidental UV exposure.

Chemical aerosols and mists: Agents such as hydrogen peroxide vapor, triethylene glycol (TEG) aerosols, and metallic nanoparticle sprays have been investigated for air disinfection. For example, during the COVID-19 pandemic, regulatory agencies permitted limited emergency use of certain glycol-based aerosols. However, chemical mists suffer from non-uniform distribution, short-lived efficacy, and, in some cases, toxicological concerns for occupants. Moreover, most of these systems operate in “shock” mode (intermittent fumigation), rather than as a continuous protective measure.

Corona discharge and plasma ionizers: Electronic ionizers generate charged ions intended to destabilize microorganisms. While conceptually promising, their performance has been limited by the following: low ion concentrations (typically 10⁴-10⁶ ions/cm³, insufficient for rapid pathogen neutralization). Production of ozone and reactive oxygen species, which are harmful to human health, and strictly regulated. Limited ability to generate stable nanoparticles in addition to ions.

Nanoparticle-based antimicrobials: It is well established in the scientific literature that metallic and mineral nanoparticles (e.g., silver, copper, titanium dioxide, zinc oxide, and iron oxide) exhibit antimicrobial activity. Mechanisms include binding to viral surface proteins, damaging lipid membranes, and interacting with microbial genomes. However, most nanoparticle studies focus on surface coatings, liquids, or therapeutic formulations, not on real-time generation and room-scale air delivery. Systems capable of producing nanoparticles at sufficiently high airborne concentrations for continuous pathogen neutralization are absent from the market.

The use of nanoparticles/ions for mitigating pathogen transmission is documented extensively in the literature. In 2021, the Environmental Protection Agency (EPA) allowed the state of Tennessee to use Triethylene Glycol (TEG) vapor and nanoparticles that are known to permeate and kill the COVID-19 virus particles by disrupting the protein and membrane structures. The technology was pioneered by Grignard Pure. Other metallic particles have long been known to have anti-viral properties. These include silver, titanium, copper, zinc oxide, and iron oxide. Among the metallic nanoparticles, silver nanoparticles have attracted the greatest attention. They can attach to viral surface glycoproteins and enter host cells, where they exhibit virucidal activity by interacting with the viral genome. The primary mechanisms by which ions deactivate viruses include disrupting the viral envelope, denaturing viral proteins, and damaging the viral genome. These actions prevent the virus from attaching to host cells, replicating, and causing infection. Metal ions and their compounds promote developing antiviral treatments and disinfectants. However, more research is needed to fully understand their mechanisms and optimize their use in clinical and environmental settings. Because of the very small size of nanoparticles, they have a high ability to cross cell membranes, reach the blood and various organs of the body. The toxicity associated with these particles is determined by their chemical composition and is an important factor to consider while determining the active ingredient for our nanoparticle generator. Bio-based antipathogenic particles typically have minimal or no side effects.

Nonetheless, conventional air purification technologies and approaches, as described above, are hindered by one or more drawbacks and unmet needs, including insufficient speed of pathogen inactivation, where current filtration and UV systems require circulation or exposure time, leaving pathogens active during transit and enabling short-range person-to-person transmission. Filtration processes are also energy-intensive, remove particles only after circulation, and do not instantly neutralize pathogens, while UV irradiation processes, in addition to requiring prolonged exposure, also consume high energy. Insufficient concentrations of neutralizing agents, where existing ionizers and misting devices produce too few ions or nanoparticles to meaningfully reduce airborne viral load in real time. Ozone and other harmful byproducts are formed when using corona and plasma ionizers, for instance, where, while generating ions, these methods also create ozone, which is a respiratory irritant and unacceptable for continuous indoor use. Corona/plasma ionizers are also disadvantaged in generating only low ion concentrations (˜10⁴-10⁶ ions/cm³), insufficient for rapid pathogen deactivation. Limited integration into continuous air handling, where many systems function only intermittently or at localized scales and do not integrate with building-scale air circulation for constant protection. Inability to simultaneously generate nanoparticles and ions, where prior art and conventional knowledge typically provide either ions (ionizers) or nanoparticles (sprays), but not a balanced mixture of both, and certainly not at ultrahigh densities. Aerosol sprays and nanoparticle mists deliver therapeutic or localized antimicrobial effects, but do not continuously protect large indoor volumes. Lack of targeted viral neutralization, while nanoparticles are known to have antimicrobial activity, no system in the art has demonstrated the ability to continuously generate nanoparticles at the correct size (<10 nm) and concentration to block viral receptor proteins and agglomerate pathogens in airborne environments.

Therefore, there is a clear and urgent need for a system that can: rapidly neutralize airborne pathogens at the point of exposure rather than merely filtering them out. Generate ultrahigh concentrations of antimicrobial nanoparticles and ions (≥1×10¹²/cm³), orders of magnitude higher than ionizers of the prior art. Produce nanoparticles of the optimal size (<10 nm) for interaction with viral receptor proteins. Simultaneously generate nanoparticles and ions in a balanced ratio (e.g., ˜50:50) to leverage both receptor-blocking and germicidal effects. Operate without ozone formation, ensuring occupant safety during continuous use. Integrate seamlessly into building HVAC systems with energy-efficient cycling between disinfection and filtration modes.

The present invention addresses the aforementioned needs and problems by providing a flame ionization nanoparticle and ion generator that overcomes the shortcomings of existing air treatment technologies and provides continuous, safe, and highly effective neutralization and inactivation of airborne pathogens. The disclosed method for airborne pathogen neutralization and inactivation employs an anti-pathogenic nanoparticle and ion generator that generates a very high concentration of nanoparticles and ions (E14, where E is the exponent of 10, per cubic centimeter or 10\hat{ }14/cm\hat{ }3 or 10¹⁴/cm³), these nanoparticles and ions so produced are present constantly in the indoor environment, and continuously inhibit and neutralize the virulence of pathogenic bioaerosols.

### SUMMARY OF THE INVENTION

The following listing of embodiments is a non-limiting statement of various aspects of the invention. Other aspects and variations will be evident in light of the entire disclosure.

The present invention provides a nanoparticle and ion generator using flame ionization of salt solutions to produce a mixture of stable nanoparticles (<10 nanometers (nm)) and ions at ultra-high concentrations or densities (≥1×10\hat{ }12 particles per cubic centimeter (cm³)) at flow rates of ≥2000 cubic feet per minute (CFM). Further, the present invention provides a method for airborne pathogen neutralization and inactivation in an enclosed environment or space by employing the flame ionization nanoparticle and ion generators configured to supply a mixture of anti-pathogenic nanoparticles and ions in enclosed environments or spaces to deactivate or neutralize airborne pathogenic microorganisms, including viruses, bacteria, and spores.

The present invention provides flame ionization-based simultaneous nanoparticle and ion generators that are capable of supplying high concentrations of natural nanoparticles (at an ultrafine size of <10 nm) and ions that bind to pathogenic microorganisms' receptors, resulting in neutralization, and/or inactivation, ultimately removal from the enclosed environment or space to stop them before they can be transmitted. The nanoparticles, aided with or without the simultaneously produced ions, generated in the system and by the method of the present invention, immediately provide a shield of protection lasting until the conventional filtration system, such as via a two-duct system regulated by a controller, purges the neutralized, inactivated, or deactivated pathogenic microorganisms along with any free or unused nanoparticles in a cycle before the next cycle of injection of freshly generated nanoparticles and ions can be injected, infused, or dispersed into the enclosed environment or space with or without new flow in of ambient air that may contain more pathogenic microorganisms to be neutralized or inactivated and removed at the end of this new cycle and hence, the system and method of the present invention works continuously and efficiently. So, in this cyclical manner, the flame ionization-based simultaneous nanoparticle and ion generators inject another batch of a mixture of nanoparticles and ions that are evenly dispersed via the injector throughout the enclosed space or environment, such as a room, and then the mixture of nanoparticles and ions, which are anti-pathogenic nanoparticles and ions generated by the generators starting from a liquid salt solution or a mixture of liquid salt solutions, bind to any remaining bioaerosols or airborne pathogenic microorganisms present in the enclosed space or environment, such as the room. The strategy in the system and method of the present invention dramatically reduces the time pathogens spend circulating throughout the air in the enclosed environment or space, such as the room, where they might have entered by intake of ambient air or even brought in by an infected carrier entering the enclosed environment or space, such as the room, thereby minimizing the chances of exposure from the beginning by terminating the possibility of transmission by removal of neutralized or inactivated airborne pathogenic microorganisms from the start. The flame ionization-based simultaneous nanoparticle and ion generators of the present invention thus provide a rapid, energy-efficient method to protect against the spread of many bioaerosols or airborne pathogenic microorganisms in any indoor area and will ultimately aid in effectively abating any such airborne pathogenic microorganism-borne diseases, including the COVID-19 pandemic.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention and are incorporated in and constitute a part of the present invention and, together with the description, serve to explain the principle of the invention. In the drawings,

FIG. 1 illustrates a representative flow chart of the system and method of a representative embodiment and example of the present invention.

FIG. 2 illustrates an embodiment of the present invention and illustrates a representative example of a flame ionization-based nanoparticle (and ion) generator of the present invention.

FIG. 3 illustrates an embodiment of the present invention and illustrates a representative example of the present invention showing stable nanoparticles as generated by the flame ionization-based nanoparticle (and ion) generator of the present invention.

FIG. 4 illustrates an embodiment of the present invention and illustrates the process involved in conventional filtration systems (top panels) versus a representative example of the process of the present invention with the flame ionization-based nanoparticle (and ion) generator of the present invention (bottom panel).

FIG. 5 illustrates an embodiment of the present invention and illustrates a representative example of the present invention, illustrating the reduction in viral load, i.e., the number of pathogens at a low concentration of nanoparticles generated with the flame ionization-based nanoparticle (and ion) generator of the present invention.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as a basis for teaching one skilled in the art to variously practice the present invention.

All illustrations of the drawings are to describe selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the term “about” refers to a rough estimate of the number or amount of the quantity referred to and is in the vicinity of the actual number or figure immediately following said term, where the actual number or figure or amount could be slightly higher or lower.

As discussed above, there remains a need in the art for an energy-efficient, ozone-free system capable of generating extremely high concentrations of nanoparticles and ions that rapidly neutralize airborne pathogens across enclosed environments.

The present invention addresses the aforementioned need and provides a nanoparticle and ion generator for the rapid neutralization of airborne pathogens in enclosed environments. The invention introduces structural, functional, and performance-based improvements that directly address deficiencies of existing air purification and pathogen-control technologies.

In an embodiment of the present invention, it provides an anti-pathogenic system comprising: a reservoir containing a liquid solution, including an aqueous salt solution; an atomizing device, including a nebulizer, configured to generate microdroplets; at least one ionization source, preferably a flame ionization burner, which converts droplets into nanoparticles and ions; an airflow system delivering the nanoparticles/ions into an enclosed environment at a volumetric flow rate of ≥2000 CFM; a controller regulating particle size, distribution, concentration, and system cycling, wherein the ionization source produces a mixture of nanoparticles and ions, wherein the mixture of nanoparticles and ions is anti-pathogenic against airborne pathogenic microorganisms in an enclosed space or environment, and wherein nanoparticles are <10 nm in diameter and the total concentration is ≥1×10¹² particles/cm³.

Although a nebulizer is preferred, the invention is not limited to this form of atomizer. In some embodiments of the present invention, as disclosed herein, the atomizer alternatively includes an ultrasonic atomizer producing fine mist droplets using piezoelectric elements, an electrohydrodynamic (EHD) atomizer generating charged droplets through electric fields, a rotary atomizer, a spinning disk or cup atomizer for higher throughput. Each atomization method may be tuned to produce sub-micron droplets which, upon ionization, yield nanoparticles <10 nm.

While flame ionization is preferred due to its ability to produce high-density nanoparticles without ozone, there are alternative embodiments. In some embodiments of the present invention, as disclosed herein, the ionization source includes thermal plasmas, including microwaves or RF plasma torches, laser-induced ionization, and hybrid flame-plasma reactors combining combustion and plasma discharge. In all such embodiments, the ionization source is configured to maintain ozone-free or near-ozone-free operation (<5 ppb ozone) to ensure occupant safety.

In some embodiments of the present invention, as disclosed herein, the liquid solution, including an aqueous salt solution, has multiple variations and embodiments. Although sodium chloride (NaCl) is a preferred salt embodiment of the aqueous salt solution for generating nanoparticles and ions due to its safety, availability, and proven antiviral efficacy, other salts and compounds may be used, including but not limited to: potassium chloride (KCl), calcium chloride (CaCl₂), magnesium chloride (MgCl₂), zinc chloride (ZnCl₂), and bio-derived salts, including amino acid-based salts, organic acid salts, or a combination thereof. Combinations or layered mixtures of salts are used to tailor nanoparticle surface chemistry for specific antimicrobial applications.

In some embodiments of the present invention, as disclosed herein, the system generates a balanced ratio (˜50:50) of nanoparticles and ions. In alternative embodiments, the ratio is deliberately shifted between a range of 70:30 to 30:70, depending on the desired antimicrobial mechanism, wherein ion-rich clouds are favored for membrane disruption of bacteria, and nanoparticle-rich clouds are favored for viral receptor blocking. Ratio control is achieved by adjusting flame temperature, droplet feed rate, or chamber airflow.

Importantly, some embodiments of the present invention, as disclosed here, the at least one ionization source, preferably the flame ionization burner, which has a single-flame burner that converts atomized droplets into nanoparticles and ions. In some other embodiments of the present invention, as disclosed here, the at least one ionization source, preferably a flame ionization burner, which has a multi-flame burner or burners where the microdroplets pass sequentially through multiple flame zones. This yields smaller particle sizes, increased ion density, and enhanced uniformity. In a two-flame embodiment of the present invention as disclosed here, the first flame produces nanoparticles in the range of 5-15 nm, while the second flame reduces the average size to <10 nm and increases ion fraction by 10-20%.

The system of the present invention, as disclosed here, is capable of being deployed in multiple configurations, including but not limited to portable standalone units, which are similar in form factor to humidifiers or purifiers, suitable for offices, schools, or medical rooms; HVAC-integrated units, which are installed in air ducts to continuously treat circulating air in large buildings; ducted two-channel systems, where one channel operating in free-flow nanoparticle mode, the other in filtration mode, alternated by a blast gate under controller regulation; and vehicle-integrated units for aircraft cabins, buses, or trains, where rapid pathogen neutralization is critical.

The system of the present invention, as disclosed here, further comprises a controller configured to monitor nanoparticle size distribution using an integrated particle counter, adjust flame intensity, airflow rate, humidity, and droplet feed cycles, operate a blast gate alternating between filtered and unfiltered ducts, and interface with building management systems for automated operation. In some other embodiments of the present invention, the controller employs machine learning algorithms to optimize parameters in real time based on sensor feedback.

In some embodiments of the present invention, as disclosed here, the nanoparticles agglomerate with pathogens into micron-sized clusters (>2 μm), which then settle by gravity or are removed by filtration. This provides a two-step defense: (i) inactivation by receptor blocking or membrane disruption, followed by (ii) removal by agglomeration and settling.

The present invention discloses a flame ionization nanoparticle/ion generator that generates natural particles<10 nm with a concentration production rate of 1 to 10¹⁰⁰ particles-ions/mL at 2000 cfm, wherein the device of the present disclosure is used to discharge the nanoparticle/ion in a closed environment that consists of pathogens, and the bombardment of the nanoparticles and ions onto the pathogens leads to the attachment of the nanoparticles and ions onto the pathogens to deactivate the pathogens for filtration, wherein the salt is the preferred material to generate nanoparticles and ions. In the present disclosure, a controller to control and monitor nanoparticle size distribution, concentration, flow rate, on/off of the gate to alternating flow, temperature, and humidity. In the present disclosure, a two-duct system that consists of two chambers with a circulating system, wherein one chamber of the two-duct system has a filter, and the other chamber of the two-duct system has no filter, and switching of the circulation between the two chambers is controlled by a blast gate, which in turn is controlled by the controller.

The present invention further provides methods for validating pathogen deactivation, including: chamber-based viral challenge tests with surrogate organisms (e.g., MS2 bacteriophage, Phi6); continuous sampling using bio-impactors and plaque assays; physical characterization by SEM, EDS, and SMPS; and comparative testing against prior art ionizers and filters, demonstrating at least 99% reduction in airborne pathogen load within 10 minutes of operation.

The present invention also discloses a method to test and optimize the efficiency of deactivation of pathogens, including nanoparticle samples to be collected by a bio pump on the surface of a fine filtration membrane, and their size, shape, and elemental composition analyzed using a scanning electron microscope. Separately, nanoparticles are generated, then injected into a 1000 ft³ stainless steel chamber with a scanning mobility particle sizer (SMPS) attached, affording continuous sample collection. The resulting SMPS data will be analyzed to ascertain the evolution of the nanoparticle size distribution over time. This analysis informs the injection cycle studies, allowing determination of the minimum nanoparticle concentration needed to ensure room saturation. The nanoparticles exhibiting ideal characteristics are selected, and their respective anti-pathogenic properties against viruses are evaluated. Firstly, a pathogen surrogate of SARS-CoV-2 (e.g., MS2 phage and Phi6) is aerosolized and then injected into a 1000 ft³ stainless steel chamber. A bio pump is used to collect the dispersed particles onto agar petri dishes coated with host bacteria every five minutes for one hour. The petri dishes are incubated and observed for 24 hours to determine the natural decay of the pathogen. Secondly, this process is to be repeated; however, nanoparticles are simultaneously injected into the chamber with the pathogen surrogate. The rate of decay in the absence and presence of the nanoparticle/ion is compared. This information is used to determine the magnitude of deactivation by each nanoparticle type.

The present invention provides systems and methods for the generation and use of a mixture of anti-pathogenic nanoparticles and ions, which have an ozone-free operation, ensuring safety in occupied spaces. Further, the concentration of harmful materials is capped or modulated by the controller to comply with occupational nanoparticle exposure guidelines. There is an optional feedback loop that disables the generator if ion and/or nanoparticle levels fall outside a safe operating range.

In an embodiment of the present invention, it provides a nanoparticle and ion generator comprising: a nebulizer configured to atomize a salt solution into droplets; a flame ionization source positioned to receive said droplets and convert them into nanoparticles and ions; an airflow system delivering the nanoparticles and ions at a volumetric flow rate of about 2000 cubic feet per minute or greater, wherein the generator produces a mixture of nanoparticles having a size less than 10 nanometers and ions, at a concentration of at least 1.0×10¹² particles per cubic centimeter, and wherein the generator operates without ozone production. In another embodiment of the present invention, as disclosed here, it provides a generator, wherein the nanoparticles and ions are produced in approximately a 1:1 ratio. In another embodiment of the present invention, as disclosed here, it provides a generator, further comprising a controller configured to monitor and adjust nanoparticle size distribution, concentration, airflow, humidity, and flame conditions. In another embodiment of the present invention, as disclosed here, it provides a generator, wherein the nebulizer is configured to produce sub-micron droplets that result in nanoparticles of less than 10 nm upon flame ionization. In another embodiment of the present invention, as disclosed here, it provides a generator, wherein the flame ionization source comprises multiple flames arranged to sequentially reduce particle size and increase ion concentration. In another embodiment of the present invention, as disclosed here, it provides a generator, wherein the nanoparticle and ion generator is integrated into a two-duct circulation system comprising: a first duct with a filtration unit; and a second duct without filtration, wherein the two-duct circulation system has a blast gate or valve that alternates circulation between the two ducts to balance pathogen deactivation with removal of deactivated organisms. In another embodiment of the present invention, as disclosed here, it provides a generator, wherein the salt comprises sodium chloride. In another embodiment of the present invention, as disclosed here, it provides a generator, wherein the nanoparticles attach to viral receptor proteins and block host cell binding. In another embodiment of the present invention, as disclosed here, it provides a generator, wherein the nanoparticles agglomerate with pathogens, increasing their settling rate.

In an embodiment of the present invention, it provides a method of reducing airborne pathogens in an enclosed environment, comprising: generating a cloud of nanoparticles and ions using the generator as disclosed herein above; dispersing said nanoparticles and ions into the enclosed environment; and deactivating airborne pathogens through receptor blocking, membrane disruption, or agglomeration caused by the nanoparticles and ions. In another embodiment of the present invention, as disclosed here, it provides a method of reducing airborne pathogens in an enclosed environment, wherein the nanoparticles attach to viral receptor proteins and block host cell binding. In another embodiment of the present invention, as disclosed here, it provides a method of reducing airborne pathogens in an enclosed environment, wherein the nanoparticles agglomerate with pathogens, increasing their settling rate. In another embodiment of the present invention, as disclosed here, it provides a method of reducing airborne pathogens in an enclosed environment, wherein dispersal occurs in coordination with a two-duct alternating filtration system. In another embodiment of the present invention, as disclosed here, it provides a method of reducing airborne pathogens in an enclosed environment, wherein the nanoparticles and ions are produced without ozone formation.

In an embodiment of the present invention, it provides a method for evaluating the efficiency of pathogen deactivation by nanoparticles and ions, comprising: nebulizing a salt solution and introducing the resulting droplets into a flame ionization field to generate nanoparticles and ions; introducing a surrogate microorganism into a test chamber; dispersing the generated nanoparticles and ions into the test chamber; sampling the microorganism over time using an impactor, and analyzing the collected samples by plaque assay; and characterizing the nanoparticles and ions using at least one of a scanning electron microscope, energy dispersive spectroscopy, or a scanning mobility particle sizer.

The present invention discloses a system for generating a mixture of nanoparticles and ions, comprising: a salt solution reservoir; a nebulizer; a mixing chamber; one or more flame ionization burners; a compressed air supply; a compressed fuel supply; and a ducted delivery system, wherein, the droplets generated from the nebulizer enter the flame zone of the one or more flame ionization burners, where thermal ionization yields nanoparticles and ions. In this system, multiple flame stages may be employed to progressively reduce particle size and increase ion density.

In an embodiment of the present invention, it provides a system for generating antimicrobial nanoparticles and ions, comprising: means for atomizing a liquid solution into droplets; means for converting said droplets into a mixture of nanoparticles and ions through flame ionization; means for delivering and dispersing the mixture of nanoparticles and ions into an enclosed environment at a concentration sufficient to deactivate airborne pathogens; and means for controlling particle size, concentration, and ion-to-nanoparticle ratio, wherein the system produces the mixture of nanoparticles and ions as a cloud at concentrations of at least 1.0×10¹² particles per cubic centimeter, wherein said nanoparticles and ions are generated at concentrations sufficient to reduce airborne pathogen viability by at least 90% within 10 minutes. In another embodiment of the present invention, providing the system for generating antimicrobial nanoparticles and ions, as disclosed herein, wherein said nanoparticles have an average size less than 10 nanometers and said system operates without producing ozone above 5 ppb. In another embodiment of the present invention, providing the system for generating antimicrobial nanoparticles and ions, as disclosed herein, wherein the ionization source comprises a flame, plasma, or laser-induced ionization device. In another embodiment of the present invention, providing the system for generating antimicrobial nanoparticles and ions, as disclosed herein, wherein the atomizing device comprises a nebulizer, ultrasonic atomizer, or rotary atomizer. In another embodiment of the present invention, providing the system for generating antimicrobial nanoparticles and ions, as disclosed herein, wherein the liquid solution comprises a salt selected from sodium chloride, potassium chloride, calcium chloride, magnesium chloride, zinc chloride, or mixtures thereof. In another embodiment of the present invention, providing the system for generating antimicrobial nanoparticles and ions, as disclosed herein, wherein the system is a portable unit, HVAC-integrated unit, or vehicle cabin unit.

In an embodiment of the present invention, it provides a method for deactivating airborne pathogens, comprising: generating nanoparticles and ions in sufficient concentration to achieve at least a 99% reduction in viable airborne pathogens within 30 minutes; and dispersing said nanoparticles and ions into an enclosed space, wherein the nanoparticles are less than 10 nanometers (nm) in size and the ions are germicidal. In another embodiment of the present invention, the method for deactivating airborne pathogens, as disclosed herein, wherein the nanoparticles block viral receptor proteins and the ions destabilize pathogen membranes. In another embodiment of the present invention, the method for deactivating airborne pathogens, as disclosed herein, wherein the nanoparticles and ions are generated without ozone production.

In an embodiment of the present invention, it provides an air treatment system for pathogen reduction, comprising: a generator configured to produce a mixture of nanoparticles and ions at a concentration of at least 1.0×10¹² per cubic centimeter; a delivery unit configured to disperse said mixture into an indoor space; and a control system configured to regulate operating conditions to maintain said concentration, wherein the nanoparticles and ions are effective to deactivate pathogens in the air. In another embodiment of the present invention, the system as disclosed herein, wherein the control system adjusts operating parameters to maintain a ratio of nanoparticles to ions between 30:70 and 70:30. In another embodiment of the present invention, the system, as disclosed herein, wherein the generator comprises at least one flame ionization source and one atomizing source, and equivalents thereof.

In an embodiment of the present invention, it provides a system for airborne pathogen reduction, comprising: a nanoparticle and ion generator configured to: generate a concentration of nanoparticles and ions sufficient to reduce viable airborne pathogen load by at least 99% within 10 minutes of operation in a 1000 ft³ chamber; maintain said concentration continuously or intermittently in coordination with a building circulation system; and operate without producing ozone.

In some embodiments of the present invention, as disclosed herein, the operation of the generator results in at least a 99% reduction of viable airborne viral load within 10 minutes of exposure in a 1000 ft³ test chamber.

In some embodiments of the present invention, as disclosed herein, the airborne pathogen load is reduced by at least 99.7% within 30 minutes of operation.

In some embodiments of the present invention, as disclosed herein, the mixture of nanoparticles and ions is produced at concentrations sufficient to achieve a 3-log reduction in viable pathogen concentration compared to natural decay.

In some embodiments of the present invention, as disclosed herein, the average nanoparticle size is maintained at less than 10 nanometers with a standard deviation of ±2 nanometers.

In some embodiments of the present invention, as disclosed herein, the total concentration of nanoparticles and ions is at least 1.0×10¹² particles per cubic centimeter at an airflow of 2000 cubic feet per minute.

In some embodiments of the present invention, as disclosed herein, the nanoparticles agglomerate into clusters of at least 2 micrometers within 15 minutes, thereby increasing gravitational settling of deactivated pathogens.

In some embodiments of the present invention, as disclosed herein, the generator produces at least 10⁶-fold greater ion concentration compared to a conventional corona discharge ionizer operated under equivalent chamber conditions.

In some embodiments of the present invention, as disclosed herein, the operation produces ozone concentrations below 5 ppb, measured during continuous operation in a 1000 ft³ chamber.

In some embodiments of the present invention, as disclosed herein, the salt in the salt solution comprises at least one of sodium chloride, potassium chloride, magnesium chloride, calcium chloride, or mixtures thereof.

In some embodiments of the present invention, as disclosed herein, the nanoparticles produced from sodium chloride exhibit antiviral efficacy against MS2 bacteriophage in controlled chamber testing.

In some embodiments of the present invention, as disclosed herein, the deployment in a vehicle cabin achieves at least 99% airborne viral load reduction within 10 minutes of generator operation.

In some embodiments of the present invention, as disclosed herein, the controller dynamically adjusts operation based on real-time measurements of nanoparticle concentration and ion density to maintain effective pathogen neutralization.

In some embodiments of the present invention, as disclosed herein, the surrogate organisms for pathogenic microorganisms, including MS2 bacteriophage, Phi6 bacteriophage, and Bacillus subtilis spores, exhibit at least 90% reduction in viability within 15 minutes of generator exposure.

In some embodiments of the present invention, as disclosed herein, the testing methods using surrogate viruses in stainless steel chambers, SMPS particle sizing, SEM/EDS imaging, and plaque assays confirm rapid decay rates and support enablement.

In an embodiment of the present invention, it provides a nanoparticle and ion generator, comprising: a reservoir containing a liquid solution, wherein the liquid solution is a salt solution; a first injector for injecting the salt solution from the reservoir to an atomizer by pressurized air; an atomizer, wherein the atomizer is a nebulizer configured to atomize the salt solution into sub-micron liquid droplets of salt; a second injector for injecting the sub-micron liquid droplets of salt generated by the nebulizer into a space containing one or more flame ionization sources by pressurized air; a space with one or more flame ionization sources positioned to receive the sub-micron liquid droplets of salt and convert them into a mixture of nanoparticles and ions of salt; an airflow system delivering the mixture of nanoparticles and ions of salt at a volumetric flow rate of at least 2000 cubic feet per minute (cfm), wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof, wherein the space with one or more flame ionization sources comprises a compressed fuel supply, a compressed air supply, the reservoir containing the salt solution, the atomizer, a mixing chamber, one or more burners, and optionally an accumulator, wherein the nanoparticle and ion generator produce nanoparticles in the mixture of nanoparticles and ions of salt having a size less than or equal to 10 nanometers (nm), wherein the nanoparticle and ion generator produce a density of at least 1.0×10\hat{ }12 nanoparticles and ions of salt per milliliter (ml) of air at 2000 cfm, and the concentration of nanoparticles of at least 1.0×10\hat{ }12 nanoparticles cubic centimeter (cc), and wherein the nanoparticle and ion generator operate without ozone production.

In another embodiment of the present invention, providing the nanoparticle and ion generator, as disclosed herein, wherein the mixture of nanoparticles and ions of salt is generated at a ratio that ranges between 70:30 to 30:70 ratio of nanoparticles to ions of salt, with the optimal ratio of 50:50 of nanoparticles to ions of salt.

In another embodiment of the present invention, providing the nanoparticle and ion generator, as disclosed herein, further comprising a controller configured to monitor and adjust nanoparticle size distribution, nanoparticle and ion concentration, airflow, humidity, droplet injection cycles, flame conditions, and temperature to ensure reproducible size distribution and nanoparticle to ion ratio and balance.

In another embodiment of the present invention, providing the nanoparticle and ion generator, as disclosed herein, wherein the nebulizer is configured to produce sub-micron droplets resulting in nanoparticles of less than or equal to 10 nm upon flame ionization.

In another embodiment of the present invention, providing the nanoparticle and ion generator, as disclosed herein, wherein the one or more flame ionization sources comprise multiple flames or burners arranged to sequentially and progressively reduce nanoparticle size and increase ion and nanoparticle concentration and density.

In an embodiment of the present invention, it provides a system for airborne pathogen neutralization and inactivation in an enclosed environment, comprising: a reservoir containing a liquid solution, wherein the liquid solution is a salt solution; a first injector for injecting the salt solution from the reservoir to an atomizer by pressurized air; an atomizer, wherein the atomizer is a nebulizer configured to atomize the salt solution into sub-micron liquid droplets of salt; a second injector for injecting the sub-micron liquid droplets of salt generated by the nebulizer into a space containing one or more flame ionization sources by pressurized air; a space with one or more flame ionization sources positioned to receive the sub-micron liquid droplets of salt and convert them into a mixture of anti-pathogenic nanoparticles and ions of salt; an enclosed environment containing airborne pathogens; a two-duct circulation system comprising a first duct without filtration, and a second duct with a filtration unit; a blast gate or valve associated with the two-duct circulation system that alternates circulation between the two ducts; and a third injector for injecting and dispersing the mixture of anti-pathogenic nanoparticles and ions of salt in the enclosed environment containing the airborne pathogens by pressurized air, wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof, wherein the airborne pathogens include viral pathogens, bacterial pathogens, bacterial spores, fungal spores, or a combination thereof, wherein the pressurized air is supplied by a compressed air supply source, wherein the space with one or more flame ionization sources comprises a compressed fuel supply, a compressed air supply, the reservoir containing the salt solution, the atomizer, a mixing chamber, one or more burners, and optionally an accumulator, wherein the third injector for injecting and dispersing the mixture of anti-pathogenic nanoparticles and ions of salt in the enclosed environment is part of an airflow system delivering the mixture of nanoparticles and ions of salt in the enclosed system at a volumetric flow rate of at least 2000 cubic feet per minute (cfm), wherein the one or more flame ionization sources produce nanoparticles in the mixture of anti-pathogenic nanoparticles and ions of salt having a size less than or equal to 10 nanometers (nm), wherein the nebulizer is configured to produce sub-micron droplets resulting in nanoparticles of less than or equal to 10 nm upon flame ionization, wherein the one or more flame ionization sources produce a density of at least 1.0×10\hat{ }12 nanoparticles and ions of salt per milliliter (ml) of air at 2000 cfm, and the concentration of nanoparticles of at least 1.0×10\hat{ }12 nanoparticles per cubic centimeter (cc), and wherein the one or more flame ionization sources operate without ozone production.

In another embodiment of the present invention, providing the system for airborne pathogen neutralization and inactivation in an enclosed environment, as disclosed herein, wherein the mixture of nanoparticles and ions of salt is generated at a ratio that ranges between 70:30 to 30:70 ratio of nanoparticles to ions of salt, with the optimal ratio of 50:50 of nanoparticles to ions of salt.

In another embodiment of the present invention, providing the system for airborne pathogen neutralization and inactivation in an enclosed environment, as disclosed herein, further comprising a controller configured to monitor and adjust nanoparticle size distribution, nanoparticle and ion concentration, airflow, humidity, droplet injection cycles, flame conditions, and temperature to ensure reproducible size distribution and nanoparticle to ion ratio and balance.

In another embodiment of the present invention, providing the system for airborne pathogen neutralization and inactivation in an enclosed environment, as disclosed herein, wherein the one or more flame ionization sources comprise multiple flames or burners arranged to sequentially and progressively reduce nanoparticle size and increase ion and nanoparticle concentration and density.

In another embodiment of the present invention, providing the system for airborne pathogen neutralization and inactivation in an enclosed environment, as disclosed herein, wherein the two-duct circulation system is regulated by the blast gate or valve that alternates circulation between the two ducts to balance pathogen deactivation of the airborne pathogens with removal of deactivated pathogens and free nanoparticles and ions in a cyclical but continuous manner, wherein the two-duct circulation system has the first duct operating to allow pathogen neutralization and inactivation of the airborne pathogens by the mixture of nanoparticles and ions of salt, and the second duct filtered to remove neutralized, inactivated pathogen of the airborne pathogens after neutralization and inactivation by the mixture of nanoparticles and ions of salt, regulated by the blast gate or valve alternating circulation between the two ducts to maximize efficiency and energy savings, and wherein the duct switching in the two-duct circulation system is regulated by the controller that alternates between filtered and unfiltered circulation for optimal pathogen neutralization and inactivation, and removal inside the enclosed environment.

In another embodiment of the present invention, providing the system for airborne pathogen neutralization and inactivation in an enclosed environment, as disclosed herein, wherein the nanoparticles from the mixture of nanoparticles and ions of salt attach to viral receptor proteins and block host cell binding, wherein the nanoparticles and ions from the mixture of nanoparticles and ions of salt agglomerate with pathogens, increasing their settling rate, and wherein the system results in neutralization and inactivation of pathogenic microorganisms in real-time in the enclosed environment.

In another embodiment of the present invention, providing the system for airborne pathogen neutralization and inactivation in an enclosed environment, as disclosed herein, wherein the enclosed environment includes but not confined to rooms, buildings, test sites, test containers, and the like.

In an embodiment of the present invention, it provides a method for airborne pathogen neutralization and inactivation in an enclosed environment with flame ionization-generated mixture of nanoparticles and ions of salt by atomization and flame ionization of a salt solution, the method comprising the steps of: preparing a salt solution in a reservoir for the salt solution; injecting the salt solution by a first injector from the reservoir to an atomizer by pressurized air; generating sub-micron liquid droplets of the salt solution by the atomizer, wherein the atomizer is a nebulizer; injecting the sub-micron liquid droplets of the salt solution generated by the nebulizer, by a second injector from the nebulizer into a space containing one or more flame ionization sources by pressurized air; generating a mixture of anti-pathogenic nanoparticles and ions of salt by the one or more flame ionization sources by moving the sub-micron liquid droplets of the salt solution through the one or more flame ionization sources that function as flame ionization nanoparticle and ion generators to convert the sub-micron liquid droplets of the salt solution into the mixture of anti-pathogenic nanoparticles and ions of salt; injecting and dispersing the mixture of anti-pathogenic nanoparticles and ions of salt in an enclosed environment containing airborne pathogens by a third injector by pressurized air; contacting airborne pathogens with the mixture of anti-pathogenic nanoparticles and ions of salt in the enclosed environment to neutralize and inactivate the airborne pathogens; and removing the neutralized and inactivated airborne pathogens from the enclosed environment by a two-duct circulation system comprising a first duct without filtration, and a second duct with a filtration unit regulated by a blast gate or valve associated with the two-duct circulation system that alternates circulation between the two ducts, wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof, wherein the airborne pathogens include viral pathogens, bacterial pathogens, bacterial spores, fungal spores, or a combination thereof, wherein the pressurized air is supplied by a compressed air supply source, wherein the space with one or more flame ionization sources comprises a compressed fuel supply, a compressed air supply, the reservoir containing the salt solution, the atomizer, a mixing chamber, one or more burners, and optionally an accumulator, wherein the third injector for injecting and dispersing the mixture of anti-pathogenic nanoparticles and ions of salt in the enclosed environment is part of an airflow system delivering the mixture of nanoparticles and ions of salt in the enclosed system at a volumetric flow rate of at least 2000 cubic feet per minute (cfm), wherein the one or more flame ionization sources produce nanoparticles in the mixture of anti-pathogenic nanoparticles and ions of salt having a size less than or equal to 10 nanometers (nm), wherein the nebulizer is configured to produce sub-micron droplets resulting in nanoparticles of less than or equal to 10 nm upon flame ionization, wherein the one or more flame ionization sources produce a density of at least 1.0×10\hat{ }12 nanoparticles and ions of salt per milliliter (ml) of air at 2000 cfm, and the concentration of nanoparticles of at least 1.0×10\hat{ }12 nanoparticles per cubic centimeter (cc), and wherein the one or more flame ionization sources operate without ozone production.

In another embodiment of the present invention, providing the method for airborne pathogen neutralization and inactivation in an enclosed environment, as disclosed herein, wherein the mixture of nanoparticles and ions of salt is generated at a ratio that ranges between 70:30 to 30:70 ratio of nanoparticles to ions of salt, with the optimal ratio of 50:50 of nanoparticles to ions of salt.

In another embodiment of the present invention, providing the method for airborne pathogen neutralization and inactivation in an enclosed environment, as disclosed herein, further comprising a controller configured to monitor and adjust nanoparticle size distribution, nanoparticle and ion concentration, airflow, humidity, droplet injection cycles, flame conditions, and temperature to ensure reproducible size distribution and nanoparticle to ion ratio and balance.

In another embodiment of the present invention, providing the method for airborne pathogen neutralization and inactivation in an enclosed environment, as disclosed herein, wherein the one or more flame ionization sources comprise multiple flames or burners arranged to sequentially and progressively reduce nanoparticle size and increase ion and nanoparticle concentration and density.

In another embodiment of the present invention, providing the method for airborne pathogen neutralization and inactivation in an enclosed environment, as disclosed herein, wherein the two-duct circulation system is regulated by the blast gate or valve that alternates circulation between the two ducts to balance pathogen deactivation of the airborne pathogens with removal of deactivated pathogens and free nanoparticles and ions in a cyclical but continuous manner, wherein the two-duct circulation system has the first duct operating to allow pathogen neutralization and inactivation of the airborne pathogens by the mixture of nanoparticles and ions of salt, and the second duct filtered to remove neutralized, inactivated pathogen of the airborne pathogens after neutralization and inactivation by the mixture of nanoparticles and ions of salt, regulated by the blast gate or valve alternating circulation between the two ducts to maximize efficiency and energy savings, and wherein the duct switching in the two-duct circulation system is regulated by the controller that alternates between filtered and unfiltered circulation for optimal pathogen neutralization and inactivation, and removal inside the enclosed environment.

In another embodiment of the present invention, providing the method for airborne pathogen neutralization and inactivation in an enclosed environment, as disclosed herein, wherein the nanoparticles from the mixture of nanoparticles and ions of salt attach to viral receptor proteins and block host cell binding, wherein the nanoparticles and ions from the mixture of nanoparticles and ions of salt agglomerate with pathogens, increasing their settling rate, and wherein the system results in neutralization and inactivation of pathogenic microorganisms in real-time in the enclosed environment.

In another embodiment of the present invention, providing the method for airborne pathogen neutralization and inactivation in an enclosed environment, as disclosed herein, wherein the enclosed environment includes but not confined to rooms, buildings, test sites, test containers, and the like.

In an embodiment of the present invention, it provides a method of testing pathogen neutralization in air, the method comprising the steps of: introducing a pathogen or pathogen surrogate into a test chamber; nebulizing a salt solution to form droplets; feeding the droplets into a flame ionization stage to generate nanoparticles and ions; injecting the nanoparticles and ions into the test chamber; sampling air from the test chamber at intervals; and analyzing the samples to determine microorganism concentration over time.

In another embodiment of the present invention, providing the method of testing pathogen neutralization in air, as disclosed herein, wherein sampling comprises measuring particle size distribution using a scanning mobility particle sizer (SMPS).

In another embodiment of the present invention, providing the method of testing pathogen neutralization in air, as disclosed herein, wherein sampling comprises analyzing particle composition using scanning electron microscopy with energy dispersive spectroscopy (SEM/EDS).

In another embodiment of the present invention, providing the method of testing pathogen neutralization in air, as disclosed herein, wherein sampling comprises capturing microorganisms on an impactor and culturing or quantifying infectivity.

In another embodiment of the present invention, providing the method of testing pathogen neutralization in air, as disclosed herein, wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof.

In another embodiment of the present invention, providing the method of testing pathogen neutralization in air, as disclosed herein, wherein the microorganisms are surrogates for airborne pathogens, including viral pathogens, bacterial pathogens, bacterial spores, fungal spores, or a combination thereof.

The present invention consists of a flame ionizer, a pump to control the rate at which the salt sample is added to the filaments. This process is precisely controlled to help ensure consistent particle size distribution. A nozzle is used to produce a uniform radial dispersion of nanoparticles whilst avoiding any agglomeration during the process. A control board and interface are used to simultaneously modifications of multiple parameters (e.g., injection cycles, current, voltage, carrier gas pressure, filaments on/off time intervals, etc. The technique has been proven to further break down the nanoparticles into ions.

Another objective is to provide a method to test the efficiency and selection of operating parameters: a) Nanoparticle samples to be collected by a bio pump on the surface of a fine filtration membrane, and their size, shape, and elemental composition analyzed using a scanning electron microscope (SEM/EDS). Separately, nanoparticles are generated, then injected into a 1000 ft3 stainless steel chamber and sized with a scanning mobility particle sizer (SMPS) attached, affording continuous sample collection. The resulting SMPS data will be analyzed to ascertain the evolution of the nanoparticle size distribution over time. This analysis informs the injection cycle studies, allowing determination of the minimum nanoparticle concentration needed to ensure room saturation. b) The nanoparticles exhibiting ideal characteristics are selected and their respective anti-pathogenic properties against viruses are evaluated. Firstly, a pathogen surrogate of SARS-CoV-2 (e.g., MS2 phage and Phi6) is aerosolized and then injected into a 1000 ft3 stainless steel chamber. A bio pump is used to collect the dispersed particles onto agar petri dishes coated with host bacteria every five minutes for one hour. The petri dishes are incubated and observed for 24 hours to determine the natural decay of the pathogen. Secondly, this process is to be repeated; however, nanoparticles are simultaneously injected into the chamber with the pathogen surrogate. The rate of decay in the absence and presence of the nanoparticle is compared. This information is used to determine the magnitude of deactivation by each nanoparticle type.

In the present invention as disclosed, it is preferred to use salt-based (e.g., NaCl) nanoparticles to potentially block viral receptors (impeding their capability to attach to host cell receptors). The nanoparticle generator is used to generate <10 nm particles of different compositions with high concentration. It is the high clouds of NaCl ions that will deactivate viral organisms and help with the attachment of salt particles to receptor proteins with no ozone generation. Ozone has been proven to be harmful to humans.

Finally, it is an objective of the present invention to achieve a ˜99% increase in the rate of decay of the pathogen in less than one second of exposure to the presence of the nanoparticles.

In the present invention, any atomizer, such as a nebulizer, is used to break up the liquid solution, such as a salt solution, into droplets, which become particles after drying. Conventionally, single or multiple units with combinations of a few types of nebulizers are used. The present invention provides a functional design that produces smaller particles and eliminates the larger particles.

In the present invention, the flame source and type are also important parts that are being optimized. The goal is to have one or more flame ionization sources that generate a 50:50 ratio of nanoparticles to ions. Since the concentration of nanoparticles and ions is very high, the ion cloud can be very effective in eliminating all types of microorganisms. The effectiveness of ions being highly germicidal has been known for a long time, and it is in use today. The present invention provides systems and methods for generating ions while eliminating ozone generation, which is otherwise inherent with conventional electronic ionizers. Also, conventional electronic ionizers do not generate enough ions to be effective in killing pathogenic microorganisms.

The present invention provides systems and methods that optimize the size of the nanoparticles to fit the receptor proteins, which is the principal part of improving the present technology. The nanoparticles from alternative compounds are being tested while keeping the characteristics of the substance unchanged, but initial steps are using salts as disclosed herein.

Further, the present invention provides systems and methods for investigating types of flame that can enhance this technology. The type of flame plays a role in breaking down the particles and generating higher concentrations of ions. Thus, the present invention discloses multiple flames in alternate embodiments to keep breaking down the nanoparticles into smaller sizes.

As shown in the following examples, the present invention provides systems and methods for the generation of a mixture of anti-pathogenic nanoparticles and ions, which, when injected into a chamber, such as a stainless-steel chamber comprising pathogen surrogates, e.g., MS2, Phi6 phages, leads to microbial decay measured by impactor plating and plaque assay. Further, nanoparticles generated by the present invention are characterized via SEM/EDS and SMPS. The data demonstrate pathogen decay rates increase by >99% within seconds of exposure.

The invention will be further explained by the following Examples, which are intended to be purely exemplary of the invention and should not be considered as limiting the invention in any way.

### EXAMPLES

The following example provides exemplary embodiments of the implantable devices and sealing halo units of the present invention.

### Example 1

In this example of the present invention, embodiments of the present invention are exemplified in the form of an experimental setup to generate exemplary nanoparticles and ions of salt from the disclosure of the present invention to eliminate microorganisms from an enclosed environment or space as disclosed herein.

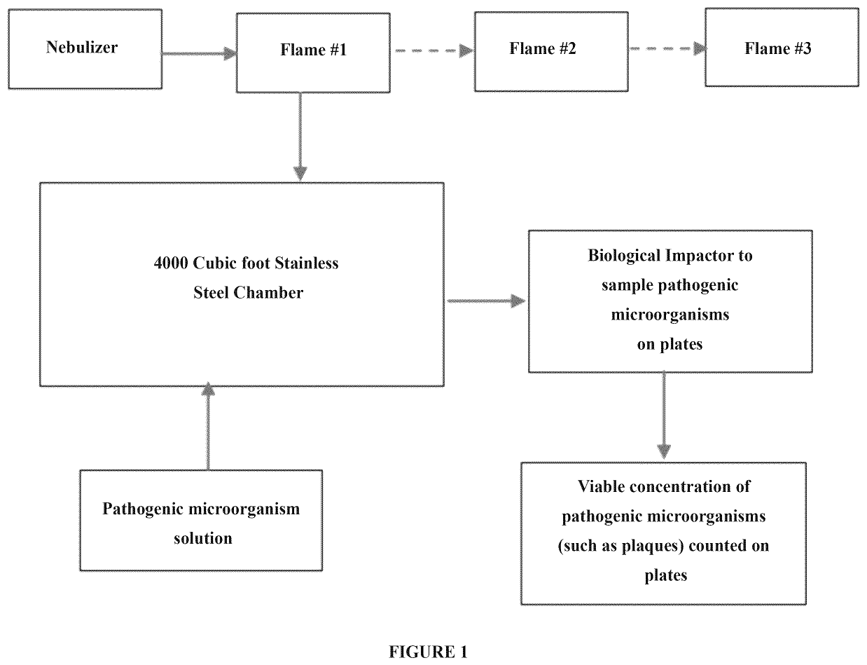

In this example, as illustrated in FIG. 1, a flowchart of the experimental setup is shown. First, a set of microorganisms, which are viruses in this experiment, is fed at 10⁵ viruses/m³ into a Chamber, which is a 4000 cubic foot stainless steel chamber. Then, the exemplary nebulizer of the present invention is turned on to produce NaCl salt droplets, which are fed to the ionization Flame #1. Next, the Flame #1 produced a mixture of antimicrobial (anti-pathogenic) nanoparticles and ions, which are then fed to the Chamber. Intermittently, at intervals of every 5 minutes, the viruses are sampled by an Impactor. The concentration of the viable viruses on the Plate is measured after 24 hours of incubation to check for viability and elimination of viruses by the mixture of nanoparticles and ions produced by the representative experimental setup of the present invention, as disclosed in this example. Further, the experimental setup may be supplemented or repeated with Flame #2, Flame #3, and so on for multiple Flames as required, to check and select the best setup for each of the airborne pathogens as a routine practice based on the present disclosure. Concentration of ions being produced, along with concentration and density of nanoparticles being produced, are measured using an ion detection counter device, and using a scanning mobility particle sizer (SMPS) for measuring particle size distribution of the nanoparticles, and using scanning electron microscopy with energy dispersive spectroscopy (SEM/EDS) for analyzing particle composition, respectively.

### Example 2

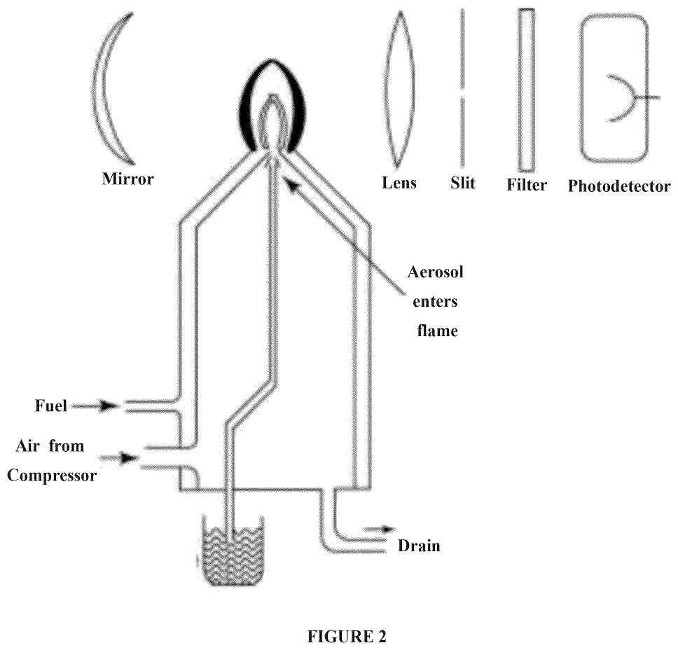

In this example of the present invention, embodiments of the present invention are exemplified in the form of a flame ionization setup as illustrated in FIG. 2, showing a representative schematic of a flame ionization-based nanoparticle (and ion) generator of the present invention. It shows the flame on a burner put on top of a mounting chamber that is also connected to an atomizer on one side, being supplied by a compressed air supply with pressurized air on one side, and being supplied by a compressed fuel supply on one side, where the atomizer connects to the sample of liquid solution, such as a salt solution on the other side. The flame is used to carry out flame ionization of droplets of the salt solution after it has undergone atomization by an atomizer, such as a nebulizer, and the flame of the present invention produces a mixture of nanoparticles and ions of salt with the help of the setup comprising a mirror, a lens, a slit, a filter, and a photodetector. Then the remnants post-generation of the mixture of nanoparticles and ions of salt leave the exemplary system of the present invention, shown in FIG. 2, by the drain.

### Example 3

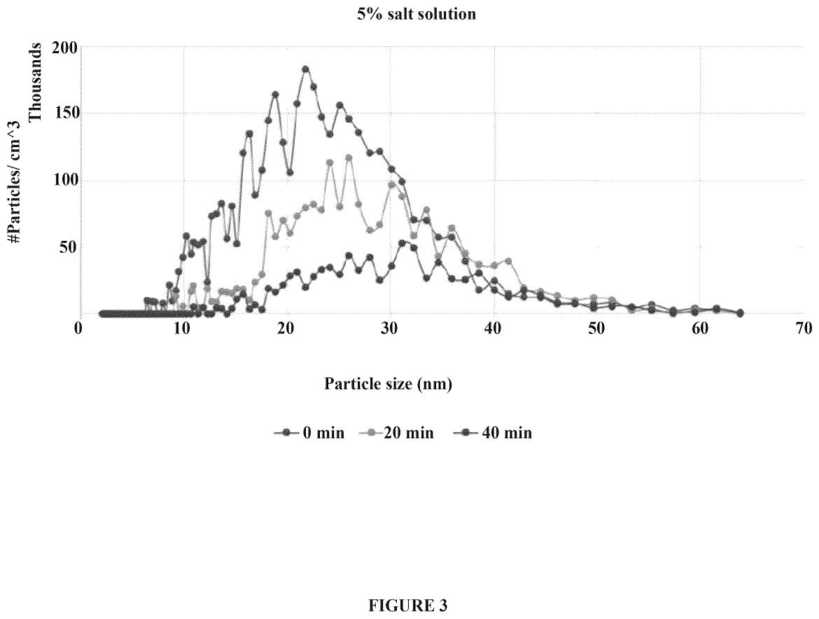

In this example of the present invention, embodiments of the present invention are exemplified in the form of an exemplary system and method for the generation of nanoparticles (and ions) starting with a 5% salt solution of NaCl. This example shows an exemplary embodiment that generates a mixture of nanoparticles and ions of salt, with the particles having an average ultrafine size of <10 nm. Here, the generated nanoparticles are studied in a 1000 ft³ stainless steel room, where 30% of the generated particles are ions and 70% of the generated particles are stable nanoparticles with no ozone generation. Since the breaking down of salt particles here generates ions, which are highly recommended for the deactivation of microorganisms, including airborne pathogens. In this example, as a preferred embodiment of the present invention, solid particles at a rate of E12 (10¹²) particles/ions/mL at 2000 cfm with nanoparticles and ions at sizes of <10 nm for deactivation of airborne pathogens are produced. The size distribution of the generated nanoparticles and ions in this example shows that they attach themselves to the receptors on the viral surface, thereby leading to viral inactivation by the nanoparticles. Here, it has also been demonstrated that stable particles are generated, as shown in FIG. 3. In addition, the high concentration of ions generated in this example of the present invention has a significant effect in deactivating viruses, where these ions are germicidal, allowing further nanoparticles to be manipulated to attach to the receptor proteins of the viruses to render them inactive. By attaching to viral receptors, nanoparticles have been demonstrated to be a promising route for viral inhibition in the present invention by this example.

### Example 4

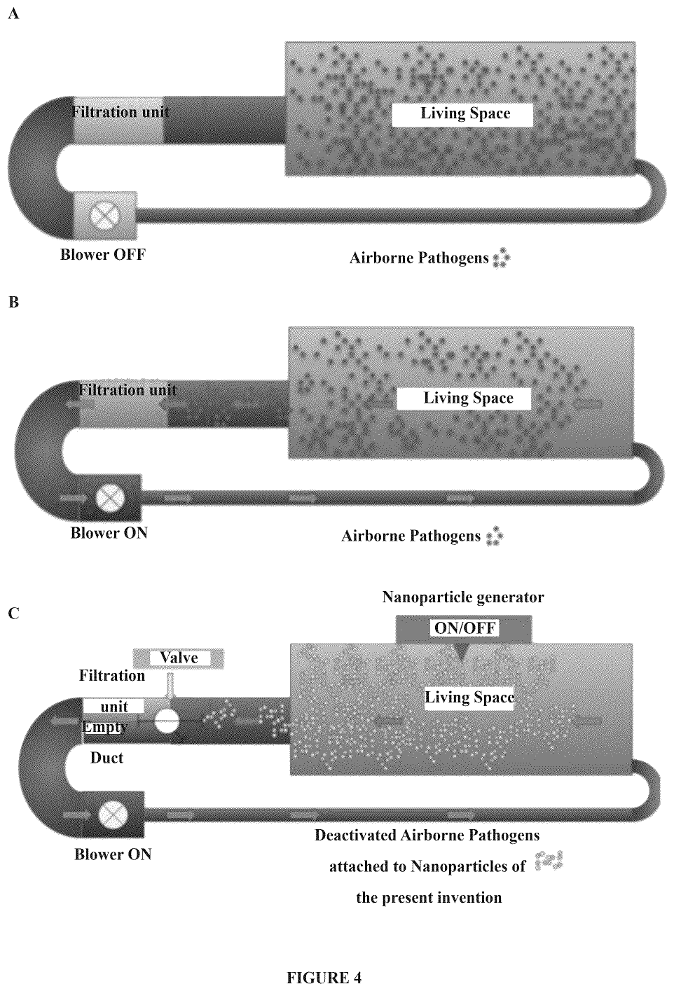

In this example of the present invention, embodiments of the present invention, as illustrated in FIG. 4 shows a schematic of the process involved in the usage of conventional filtration systems is shown compared to the present invention. Conventional systems include only the removal of pathogens through methods such as high efficiency particulate air (HEPA) filters or passing it through an ultraviolet (UV) unit. Both methods require constant energy consumption. These methods will not be able to prevent the transfer of pathogens from person-to-person over short distances, for example, in the event of sneezing or to deactivate the virus instantly throughout the building. The present innovative nanoparticle generator is to neutralize the pathogens in a room instantly. As shown in the bottom figure, there is a constant collision of nanoparticles/ions with pathogens in the air. This neutralizes them based on the inert antimicrobial/antiviral properties of the nanoparticles/ions. Even over short distances, the transmission rate of active pathogens drops significantly. There is no need for the filtration system to run continuously. Instead, the filtration system and nanoparticle generator will turn on and off for short periods of time to purge the space from deactivated pathogens and fill the room with a fresh batch of nanoparticles. This will result in a significant drop in energy consumption when compared with conventional filtration systems. Moreover, a 2-duct system can be used to improve the efficiency of operation. It consists of one with a filter and one without to allow nanoparticles/ions suspended in the air once a sufficient concentration is achieved. Based on the size of the building, air is recirculating without a filter for nanoparticles/ions to deactivate the pathogen. The blast gate will periodically switch the system to filtration mode to remove the deactivated organisms and nuisance particles.

The flame ionization nanoparticle/ion generator can be placed as a stationary unit or in a duct system in any building. The generator produces the nanoparticles/ions with simple and discrete operations, such as the operation of a humidifier or air purifier.

The essential device is the nanoparticle generator that utilizes flame ionization technology to generate natural nanoparticles (<10 nm). Since ions are germicidal, they will deactivate the virus and, at the same time, possibly mediate the attachment of nanoparticles to receptor proteins by allowing particles to be manipulated to attach receptor proteins and render them inactive. By attaching to viral receptors, nanoparticles have been demonstrated to be a promising route for viral inhibition. A prototype has demonstrated that a nanoparticle generator can produce solid particles at a rate of 10¹² particles/mL at 2000 cfm.

### Example 5

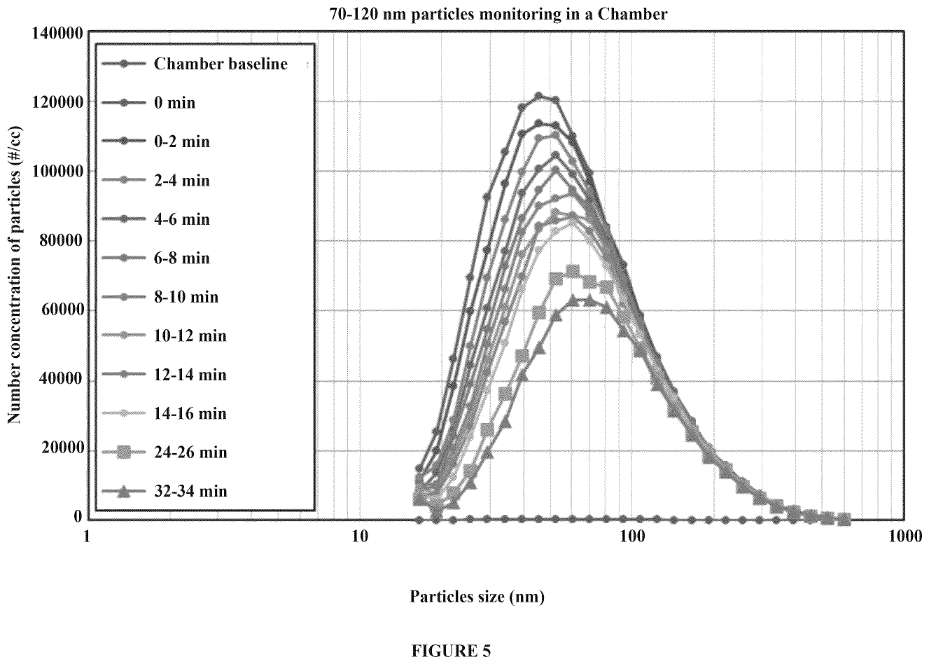

In this example of the present invention, embodiments of the present invention, as illustrated in FIG. 5, show a graphical reduction of viral load at low concentrations of nanoparticles of the present invention as monitored with exemplary nanoparticles of ultrafine sizes in the range of 70-120 nm as monitored in a chamber of EXAMPLE 1 with an exemplary microorganism, MS2 phage.

Advantages of the present invention disclosure over prior art:

The prior art provides that HEPA filters and UV irradiation remove or deactivate pathogens only after extended circulation or direct exposure, allowing pathogens to persist in the breathing zone. The generator of the present invention produces nanoparticles and ions at ultrahigh concentrations (≥1×10¹²/cm³) and disperses them uniformly into the air. Pathogens are neutralized in situ within seconds, preventing transmission before filtration.

The prior art provides that electronic ionizers typically generate ≤10⁶ ions/cm³, far too low to deactivate viruses. Aerosol sprays produce droplets that are transient and non-uniform. The present invention provides that by using flame ionization of salt aerosols, the present system and methods reliably generate 10⁶ to 10⁸ times higher concentrations of nanoparticles (plus ions) than prior art. The high-density cloud ensures statistically certain collisions between nanoparticles/ions and pathogens, achieving rapid neutralization.

The prior art provides that corona discharge and plasma ionizers generate ozone, a hazardous byproduct, limiting continuous safe use indoors. The flame ionization process as disclosed in the present invention produces no detectable ozone (<1 ppb) while still generating large quantities of germicidal ions, making it safe for continuous occupant exposure.

The systems of the prior art provide either nanoparticles (e.g., metallic sprays) or ions (ionizers), but not a controlled balance of both. The disclosed system and methods of the present invention produce a balanced mixture (˜50:50) of nanoparticles and ions, wherein the nanoparticles (<10 nm) of the present disclosure plug viral receptor proteins and agglomerate pathogens, and the simultaneously produced ions of the present invention disrupt membranes and capsids, providing an independent germicidal effect. This synergistic action yields faster and more complete neutralization.

The systems (filters, UV, ionizers) of the prior art consume energy continuously, require dedicated infrastructure, and do not optimize between disinfection and filtration. The present invention incorporates a dual-duct circulation system: one duct allows free circulation of nanoparticles/ions for pathogen neutralization, and the other duct includes a filter to remove deactivated organisms. Further, a controller-operated blast gate alternates between modes, drastically reducing energy use while maintaining pathogen control.

The systems of the prior art do not tune nanoparticle size for biological receptor targeting, nor do they control concentration dynamically. The system and methods of the present invention include a controller to adjust flame intensity, droplet injection, airflow, and humidity, ensuring particles remain <10 nm and at desired concentrations. Multi-flame configurations may be used to further refine particle size and increase ion yield.

The devices of the prior art often lack rigorous biological testing under controlled conditions. The present invention discloses a comprehensive test method: pathogen surrogates introduced into stainless steel chambers. Nanoparticles and ions of the present invention are dispersed, then measured by SEM, EDS, SMPS, and plaque assays. Data confirm >99% viral reduction within minutes, providing reproducible proof of efficacy.

The present invention thus delivers systems and methods for continuous real-time neutralization of airborne pathogens. They show orders of magnitude improvement in nanoparticle/ion density compared to prior art. They provide a dual mechanism of action in terms of nanoparticle-mediated receptor blocking and ion-mediated membrane disruption. It provides an ozone-free, safe operation. It is a means for energy-efficient integration into building systems. As shown herein, it is a validated testing protocol demonstrating rapid efficacy. Accordingly, the invention provides a novel, non-obvious, and industrially applicable solution to airborne pathogen control that overcomes every critical limitation in the art.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A nanoparticle and ion generator, comprising:
a reservoir containing a liquid solution, wherein the liquid solution is a salt solution;
a first injector for injecting the salt solution from the reservoir to an atomizer by pressurized air;
an atomizer, wherein the atomizer is a nebulizer configured to atomize the salt solution into sub-micron liquid droplets of salt;
a second injector for injecting the sub-micron liquid droplets of salt generated by the nebulizer into a space containing one or more flame ionization sources by pressurized air;
a space with one or more flame ionization sources positioned to receive the sub-micron liquid droplets of salt and convert them into a mixture of nanoparticles and ions of salt;
an airflow system delivering the mixture of nanoparticles and ions of salt at a volumetric flow rate of at least 2000 cubic feet per minute (cfm),
wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof, wherein the space with one or more flame ionization sources comprises a compressed fuel supply, a compressed air supply, the reservoir containing the salt solution, the atomizer, a mixing chamber, one or more burners, and optionally an accumulator,
wherein the nanoparticle and ion generator produce nanoparticles in the mixture of nanoparticles and ions of salt having a size less than or equal to 10 nanometers (nm),
wherein the nanoparticle and ion generator produce a density of at least 1.0×10\hat{ }12 nanoparticles and ions of salt per milliliter (ml) of air at 2000 cfm, and the concentration of nanoparticles of at least 1.0×10\hat{ }12 nanoparticles cubic centimeter (cc), and
wherein the nanoparticle and ion generator operate without ozone production.

2. The nanoparticle and ion generator of claim 1, wherein the mixture of nanoparticles and ions of salt is generated at a ratio that ranges between 70:30 to 30:70 ratio of nanoparticles to ions of salt, with the optimal ratio of 50:50 of nanoparticles to ions of salt.

3. The nanoparticle and ion generator of claim 1, further comprising a controller configured to monitor and adjust nanoparticle size distribution, nanoparticle and ion concentration, airflow, humidity, droplet injection cycles, flame conditions, and temperature to ensure reproducible size distribution and nanoparticle to ion ratio and balance.

4. The nanoparticle and ion generator of claim 1, wherein the nebulizer is configured to produce sub-micron droplets resulting in nanoparticles of less than or equal to 10 nm upon flame ionization.

5. The nanoparticle and ion generator of claim 1, wherein the one or more flame ionization sources comprise multiple flames or burners arranged to sequentially and progressively reduce nanoparticle size and increase ion and nanoparticle concentration and density.

6. A system for airborne pathogen neutralization and inactivation in an enclosed environment, comprising:
a reservoir containing a liquid solution, wherein the liquid solution is a salt solution;
a first injector for injecting the salt solution from the reservoir to an atomizer by pressurized air;
an atomizer, wherein the atomizer is a nebulizer configured to atomize the salt solution into sub-micron liquid droplets of salt;
a second injector for injecting the sub-micron liquid droplets of salt generated by the nebulizer into a space containing one or more flame ionization sources by pressurized air;
a space with one or more flame ionization sources positioned to receive the sub-micron liquid droplets of salt and convert them into a mixture of anti-pathogenic nanoparticles and ions of salt;
an enclosed environment containing airborne pathogens;
a two-duct circulation system comprising a first duct without filtration, and a second duct with a filtration unit;
a blast gate or valve associated with the two-duct circulation system that alternates circulation between the two ducts; and
a third injector for injecting and dispersing the mixture of anti-pathogenic nanoparticles and ions of salt in the enclosed environment containing the airborne pathogens by pressurized air,
wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof, wherein the airborne pathogens include viral pathogens, bacterial pathogens, bacterial spores, fungal spores, or a combination thereof,
wherein the pressurized air is supplied by a compressed air supply source,
wherein the space with one or more flame ionization sources comprises a compressed fuel supply, a compressed air supply, the reservoir containing the salt solution, the atomizer, a mixing chamber, one or more burners, and optionally an accumulator,
wherein the third injector for injecting and dispersing the mixture of anti-pathogenic nanoparticles and ions of salt in the enclosed environment is part of an airflow system delivering the mixture of nanoparticles and ions of salt in the enclosed system at a volumetric flow rate of at least 2000 cubic feet per minute (cfm),
wherein the one or more flame ionization sources produce nanoparticles in the mixture of anti-pathogenic nanoparticles and ions of salt having a size less than or equal to 10 nanometers (nm),
wherein the nebulizer is configured to produce sub-micron droplets resulting in nanoparticles of less than or equal to 10 nm upon flame ionization,
wherein the one or more flame ionization sources produce a density of at least 1.0×10\hat{ }12 nanoparticles and ions of salt per milliliter (ml) of air at 2000 cfm, and the concentration of nanoparticles of at least 1.0×10\hat{ }12 nanoparticles per cubic centimeter (cc), and
wherein the one or more flame ionization sources operate without ozone production.

7. The system for airborne pathogen neutralization and inactivation in an enclosed environment of claim 6, wherein the mixture of nanoparticles and ions of salt is generated at a ratio that ranges between 70:30 to 30:70 ratio of nanoparticles to ions of salt, with the optimal ratio of 50:50 of nanoparticles to ions of salt.

8. The system for airborne pathogen neutralization and inactivation in an enclosed environment of claim 6, further comprising a controller configured to monitor and adjust nanoparticle size distribution, nanoparticle and ion concentration, airflow, humidity, droplet injection cycles, flame conditions, and temperature to ensure reproducible size distribution and nanoparticle to ion ratio and balance.

9. The system for airborne pathogen neutralization and inactivation in an enclosed environment of claim 6, wherein the one or more flame ionization sources comprise multiple flames or burners arranged to sequentially and progressively reduce nanoparticle size and increase ion and nanoparticle concentration and density.

10. The system for airborne pathogen neutralization and inactivation in an enclosed environment of claim 6, wherein the two-duct circulation system is regulated by the blast gate or valve that alternates circulation between the two ducts to balance pathogen deactivation of the airborne pathogens with removal of deactivated pathogens and free nanoparticles and ions in a cyclical but continuous manner, wherein the two-duct circulation system has the first duct operating to allow pathogen neutralization and inactivation of the airborne pathogens by the mixture of nanoparticles and ions of salt, and the second duct filtered to remove neutralized, inactivated pathogen of the airborne pathogens after neutralization and inactivation by the mixture of nanoparticles and ions of salt, regulated by the blast gate or valve alternating circulation between the two ducts to maximize efficiency and energy savings, and wherein the duct switching in the two-duct circulation system is regulated by the controller that alternates between filtered and unfiltered circulation for optimal pathogen neutralization and inactivation, and removal inside the enclosed environment.

11. The system for airborne pathogen neutralization and inactivation in an enclosed environment of claim 6, wherein the nanoparticles from the mixture of nanoparticles and ions of salt attach to viral receptor proteins and block host cell binding, wherein the nanoparticles and ions from the mixture of nanoparticles and ions of salt agglomerate with pathogens, increasing their settling rate, and wherein the system results in neutralization and inactivation of pathogenic microorganisms in real-time in the enclosed environment.

12. A method for airborne pathogen neutralization and inactivation in an enclosed environment with flame ionization-generated mixture of nanoparticles and ions of salt by atomization and flame ionization of a salt solution, the method comprising the steps of:
preparing a salt solution in a reservoir for the salt solution;
injecting the salt solution by a first injector from the reservoir to an atomizer by pressurized air;
generating sub-micron liquid droplets of the salt solution by the atomizer, wherein the atomizer is a nebulizer;
injecting the sub-micron liquid droplets of the salt solution generated by the nebulizer, by a second injector from the nebulizer into a space containing one or more flame ionization sources by pressurized air;
generating a mixture of anti-pathogenic nanoparticles and ions of salt by the one or more flame ionization sources by moving the sub-micron liquid droplets of the salt solution through the one or more flame ionization sources that function as flame ionization nanoparticle and ion generators to convert the sub-micron liquid droplets of the salt solution into the mixture of anti-pathogenic nanoparticles and ions of salt;
injecting and dispersing the mixture of anti-pathogenic nanoparticles and ions of salt in an enclosed environment containing airborne pathogens by a third injector by pressurized air;
contacting airborne pathogens with the mixture of anti-pathogenic nanoparticles and ions of salt in the enclosed environment to neutralize and inactivate the airborne pathogens; and
removing the neutralized and inactivated airborne pathogens from the enclosed environment by a two-duct circulation system comprising a first duct without filtration, and a second duct with a filtration unit regulated by a blast gate or valve associated with the two-duct circulation system that alternates circulation between the two ducts,
wherein the salt in the salt solution is selected from a group of salts comprising sodium chloride (NaCl), potassium chloride (KCl), calcium chloride (CaCl₂), magnesium chloride (MgCl₂), zinc chloride (ZnCl₂), and bio-derived salts, including amino acid-based salts, organic acid salts, or any combination thereof, wherein the airborne pathogens include viral pathogens, bacterial pathogens, bacterial spores, fungal spores, or a combination thereof,
wherein the pressurized air is supplied by a compressed air supply source,
wherein the space with one or more flame ionization sources comprises a compressed fuel supply, a compressed air supply, the reservoir containing the salt solution, the atomizer, a mixing chamber, one or more burners, and optionally an accumulator,
wherein the third injector for injecting and dispersing the mixture of anti-pathogenic nanoparticles and ions of salt in the enclosed environment is part of an airflow system delivering the mixture of nanoparticles and ions of salt in the enclosed system at a volumetric flow rate of at least 2000 cubic feet per minute (cfm),
wherein the one or more flame ionization sources produce nanoparticles in the mixture of anti-pathogenic nanoparticles and ions of salt having a size less than or equal to 10 nanometers (nm),
wherein the nebulizer is configured to produce sub-micron droplets resulting in nanoparticles of less than or equal to 10 nm upon flame ionization,
wherein the one or more flame ionization sources produce a density of at least 1.0×10\hat{ }12 nanoparticles and ions of salt per milliliter (ml) of air at 2000 cfm, and the concentration of nanoparticles of at least 1.0×10\hat{ }12 nanoparticles per cubic centimeter (cc), and
wherein the one or more flame ionization sources operate without ozone production.

13. The method for airborne pathogen neutralization and inactivation in an enclosed environment of claim 12, wherein the mixture of nanoparticles and ions of salt is generated at a ratio that ranges between 70:30 to 30:70 ratio of nanoparticles to ions of salt, with the optimal ratio of 50:50 of nanoparticles to ions of salt.

14. The method for airborne pathogen neutralization and inactivation in an enclosed environment of claim 12, further comprising a controller configured to monitor and adjust nanoparticle size distribution, nanoparticle and ion concentration, airflow, humidity, droplet injection cycles, flame conditions, and temperature to ensure reproducible size distribution and nanoparticle to ion ratio and balance.

15. The method for airborne pathogen neutralization and inactivation in an enclosed environment of claim 12, wherein the one or more flame ionization sources comprise multiple flames or burners arranged to sequentially and progressively reduce nanoparticle size and increase ion and nanoparticle concentration and density.

16. The method for airborne pathogen neutralization and inactivation in an enclosed environment of claim 12, wherein the two-duct circulation system is regulated by the blast gate or valve that alternates circulation between the two ducts to balance pathogen deactivation of the airborne pathogens with removal of deactivated pathogens and free nanoparticles and ions in a cyclical but continuous manner, wherein the two-duct circulation system has the first duct operating to allow pathogen neutralization and inactivation of the airborne pathogens by the mixture of nanoparticles and ions of salt, and the second duct filtered to remove neutralized, inactivated pathogen of the airborne pathogens after neutralization and inactivation by the mixture of nanoparticles and ions of salt, regulated by the blast gate or valve alternating circulation between the two ducts to maximize efficiency and energy savings, and wherein the duct switching in the two-duct circulation system is regulated by the controller that alternates between filtered and unfiltered circulation for optimal pathogen neutralization and inactivation, and removal inside the enclosed environment.

17. The method for airborne pathogen neutralization and inactivation in an enclosed environment of claim 12, wherein the nanoparticles from the mixture of nanoparticles and ions of salt attach to viral receptor proteins and block host cell binding, wherein the nanoparticles and ions from the mixture of nanoparticles and ions of salt agglomerate with pathogens, increasing their settling rate, and wherein the system results in neutralization and inactivation of pathogenic microorganisms in real-time in the enclosed environment.
